(19) United States
(12) Patent Application Publication
(10) Pub. No.: US 2026/0192039 A1
(43) Pub. Date: Jul. 9, 2026

(54) APPORTION STRUCTURE FOR APPLICATION OF GELATINOUS OR VISCOUS SOLUTION

(71) Applicant: Fox Valley Innovations, LLC, Neenah, WI (US)

(72) Inventor: Robert J. Millay, Neenah, WI (US)

(73) Assignee: Fox Valley Innovations, LLC, Neenah, WI (US)

(21) Appl. No.: 19/552,743

(22) Filed: Feb. 27, 2026

Related U.S. Application Data
(63) Continuation of application No. 19/302,689, filed on Aug. 18, 2025.
Application No. 19/302,689 is a continuation-in-part of application No. 29/941,695, filed on May 10, 2024, now Pat. No. D1106818.
(60) Provisional application No. 63/687,162, filed on Aug. 26, 2024.

(51) Int. Cl. A61M 3/02; A61M 31/00

(52) U.S. Cl. CPC A61M 3/0279; A61M 31/00

(57) ABSTRACT

A method of applying a gelatinous or viscous solution and an apportion structure that has a base portion and an application portion. The base portion has an outer surface and a sidewall which form a base cavity. The application portion forms a hollow channel having a central axis and extending through the base portion generally perpendicular to the outer surface. The application portion has a first open end extending into the base cavity and having at least one attachment structure. The application portion further has a closed nipple portion extending outward from the outer surface of the base portion. The nipple portion has at least one opening therethrough.

### CROSS-REFERENCE TO RELATED APPLICATIONS

This application is a continuation of U.S. Non-Provisional patent application Ser. No. 19/302,689, filed on Aug. 18, 2025, which claims priority to U.S. Provisional Application No. 63/687,162, filed on Aug. 26, 2024, and U.S. Design application Ser. No. 29/941,695, hereby incorporated in their entireties.

### TECHNICAL FIELD

The present technology relates to structures for applying a gelatinous or viscous solution, specifically a gelatinous or viscous solution to a body cavity or injury site when targeted delivery is desired (e.g., a nostril or ear cavity or other formed body cavity).

### BACKGROUND

When applying a gelatinous or viscous solution to a body cavity or injury site, it is generally desirable to apply the solution as evenly as possible. Gelatinous or viscous solutions are applied, for example, when removing hair from a cavity, such as a nostril or ear cavity, using a waxing method, and it is desirable to apply the gelatinous or viscous solution (e.g., melted wax) as evenly as possible to ensure all undesired hairs are in contact with the solution. Gelatinous or viscous solutions are also applied to body cavities and injury sites, such as puncture or lacerations, in the form of medications (e.g., ointments, creams, etc.), and it is desirable to apply the gelatinous or viscous solution as evenly as possible to ensure equal distribution within the body cavity or injury site.

Current methods of applying the gelatinous or viscous solution have several drawbacks. Many applicators are simply sticks or other similar structures that are dipped into the gelatinous or viscous solution and placed in the desired cavity or applicators that simply drip or ooze the solution into the body cavity or injury site. There is no way to ensure the same amount of solution is applied each time. Further, there is no way to ensure the solution is applied equally about the applicator. These application structures often require massaging or manually moving the solution around to get adequate coverage within the cavity or injury site.

### BRIEF SUMMARY

The following presents a simplified summary in order to provide a basic understanding of some aspects of the disclosed subject matter. This summary is not an extensive overview. It is not intended to either identify key or critical elements or to delineate the scope of the claimed subject matter. Its sole purpose is to present some concepts in a simplified form as a prelude to the more detailed description that is presented later.

Disclosed herein is an apportion structure. In an embodiment, the apportion structure comprises a base portion having an outer surface and a sidewall which form a base cavity; and an application portion forming a hollow channel having a central axis and extending through the base portion generally perpendicular to the outer surface, wherein the application portion has a first open end extending into the base cavity and having at least one attachment structure, wherein the application portion further has a closed nipple portion extending outward from the outer surface of the base portion, and wherein the nipple portion has at least one opening therethrough.

In an embodiment, the nipple portion has greater than one opening therethrough. In a further embodiment, the nipple portion has at least three openings therethrough.

In an embodiment, each of the at least one opening is oval-shaped. In another embodiment, each of the at least one opening has a smooth inner surface. In an embodiment, the nipple portion has a wall thickness, and the inner surface of each of the at least one opening has a width equal to the wall thickness of the nipple portion along the entirety of the inner surface. In another embodiment, the nipple portion has a wall thickness, and the inner surface of each of the at least one opening has a width equal to the wall thickness of the nipple portion along at least a portion of the inner surface.

In an embodiment, the at least one attachment structure is configured to engage a structure holding a contained volume of gelatinous or viscous solution. In an embodiment, the at least one attachment structure is on an outer surface of the first end of the application portion. In another embodiment, the at least one attachment structure is on an inner surface of the first end of the application portion. In an embodiment, the at least one attachment structure is configured to engage a structure selected from the group consisting of a pump, a rubber tube, a plastic tube, a syringe, and combinations thereof.

In an embodiment, the base portion is generally round.

Disclosed herein is another embodiment of an apportion structure. In an embodiment, the apportion structure comprises a base portion having a circular outer surface and a sidewall extending at a length from the outer surface to form a base cavity, wherein a diameter of the circular outer surface is greater than a length of the sidewall; and an application portion forming a hollow channel having a central axis and extending through the base portion generally perpendicular to the outer surface, wherein the application portion has a first open end extending into the base cavity and having at least one attachment structure, wherein the application portion further has a closed nipple portion extending outward from the outer surface of the base portion, and wherein the nipple portion has at least three openings therethrough, wherein each of the at least three openings has a smooth inner surface.

In an embodiment, each of the at least three openings is oval-shaped. In an embodiment, each of the at least three openings has a first dimension in a short direction of 3.3 mm to 3.6 mm and a second dimension in a long direction of 4.3 mm to 4.6 mm.

In an embodiment, the at least one attachment structure is configured to engage a structure holding a contained volume.

Disclosed herein is a method of applying a gelatinous or viscous solution to a body cavity or injury site. In an embodiment, the method comprises providing an apportion structure, wherein the apportion structure comprises a base portion having an outer surface and a sidewall which form a base cavity, and an application portion forming a hollow channel having a central axis and extending through the base portion generally perpendicular to the outer surface, wherein the application portion has a first open end extending into the base cavity and having at least one attachment structure, wherein the application portion further has a closed nipple portion extending outward from the outer surface of the base portion, and wherein the nipple portion has at least three openings therethrough; securing the apportion structure to a contained volume of the gelatinous or viscous solution using the at least one attachment structure; inserting the nipple portion into the body cavity or injury site; and forcing the gelatinous or viscous solution through the hollow channel and out the nipple portion and into the body cavity or injury site.

In an embodiment, the step of securing the apportion structure to a contained volume of the gelatinous or viscous solution using the at least one attachment structure comprises engaging, with the at least one attachment structure, a structure selected from the group consisting of a pump, a rubber tube, a plastic tube, a syringe, and combinations thereof.

In an embodiment, the step of securing the apportion structure to a contained volume of the gelatinous or viscous solution using the at least one attachment structure comprises engaging, with the at least one attachment structure, a syringe containing a volume of the gelatinous or viscous solution, and wherein the step of forcing the gelatinous or viscous solution through the hollow channel and out the nipple portion comprises depressing a syringe plunger thereby causing the volume of the gelatinous or viscous solution to be injected into the body cavity or injury site via the at least three openings.

In an embodiment, the body cavity or injury site is a body cavity, and the method further comprises the steps of allowing the gelatinous or viscous solution to set within the cavity while the nipple portion still engages the cavity; and removing the apportion structure with set gelatinous or viscous solution from the cavity.

In an embodiment, the body cavity or injury site is a nostril or ear cavity, and the method further comprises the steps of allowing the gelatinous or viscous solution to set within the nostril or ear cavity while the nipple portion still engages the nostril or ear cavity; and removing (i) the apportion structure with set gelatinous or viscous solution from the nostril or ear cavity and (ii) cavity hairs captured in the set gelatinous or viscous solution.

To the accomplishment of the foregoing and related ends, certain illustrative aspects of the claimed subject matter are described herein in connection with the following description and the annexed drawings. These aspects are indicative of various ways in which the subject matter may be practiced, all of which are intended to be within the scope of the claimed subject matter. Other advantages and novel features may become apparent from the following detailed description when considered in conjunction with the drawings.

### BRIEF DESCRIPTION OF THE DRAWINGS

The systems, devices and methods may be better understood by referring to the following description in conjunction with the accompanying drawings, in which like numerals indicate like structural elements and features in various figures. The components in the figures are not necessarily to scale, and simply illustrate the principles of the systems, devices and methods. The accompanying drawings illustrate only possible embodiments of the systems, devices and methods and are therefore not to be considered limiting in scope.

FIG. 1 is a top front left side perspective view of an apportion structure in accordance with embodiments of the present disclosure;

FIG. 2 is a right side view thereof;

FIG. 3 is a left side view thereof;

FIG. 4 is a front side view thereof;

FIG. 5 is a back side view thereof;

FIG. 6 is a top side view thereof;

FIG. 7 is a bottom side view thereof;

FIG. 8 is a bottom left side perspective view thereof; and

FIG. 9 is a bottom right side perspective view thereof.

### DETAILED DESCRIPTION

Aspects of the system and methods are described below with reference to illustrative embodiments. The references to illustrative embodiments below are not made to limit the scope of the claimed subject matter. Instead, illustrative embodiments are used to aid in the description of various aspects of the systems and methods. The description, made by way of example and reference to illustrative reference is not meant to be limiting as regards any aspect of the claimed subject matter.

Unless stated to the contrary, implicit from context, or customary in the art, all parts and percents are based on volume. The terms “comprising,” “including,” “having” and their derivatives are not intended to exclude the presence of any additional component, step or procedure, whether or not the same is specifically disclosed. In order to avoid any doubt, all compositions claimed through the use of the term “comprising” may include any additional additive, adjuvant, compound, or structure unless stated to the contrary. In contrast, the term “consisting essentially of” excludes from the scope of any succeeding recitation any other component, step, or procedure, excepting those that are not essential to operability. The term “consisting of” excludes any component, step, or procedure not specifically delineated or listed.

As used herein, the term “body cavity or injury site” refers to an opening in an animal body, including a human, whether naturally occurring or the result of an injury. Exemplary body cavities or injury sites include nasal cavities, ear cavities, the mouth, lacerations, incisions, punctures, and the like.

The numerical ranges in this disclosure include all values from, and including, the lower and upper value. For ranges containing explicit values (e.g., a range from 1, or 2, or 3 to 5, or 6, or 7), any subrange between any two explicit values is included (e.g., the range 1-7 above includes subranges 1 to 5; 1 to 6; 1 to 7; 2 to 5; 2 to 6; 2 to 7; 3 to 5; 3 to 6; 3 to 7; etc.).

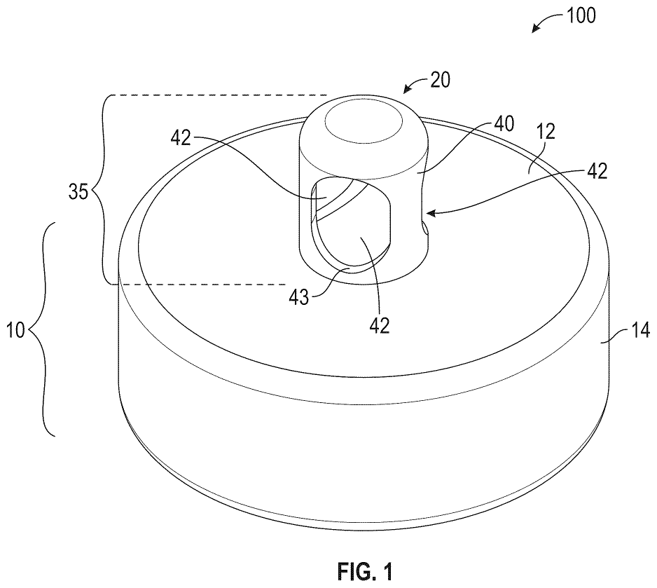

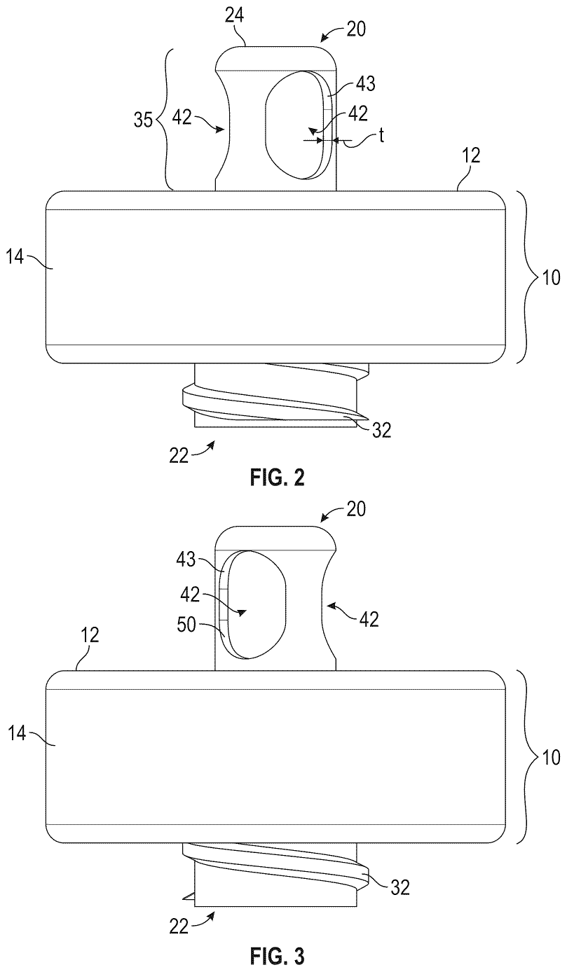

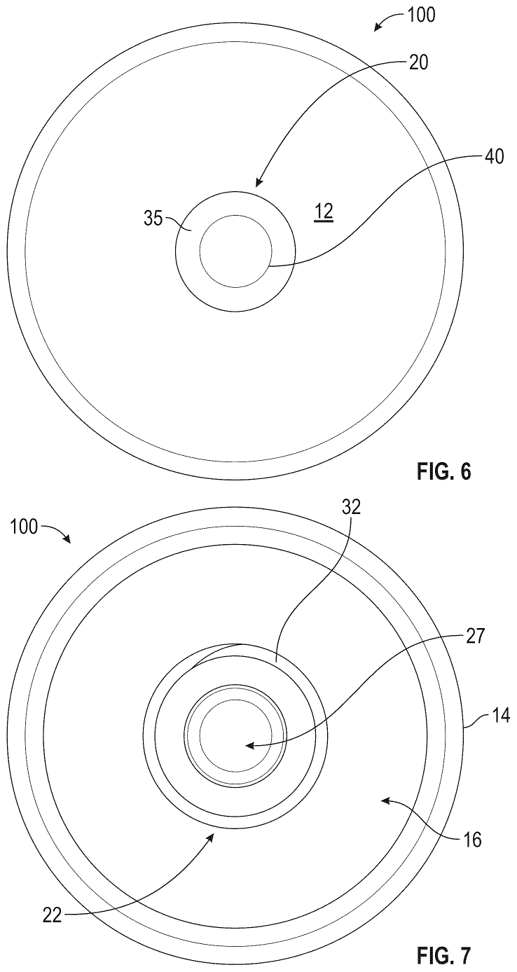

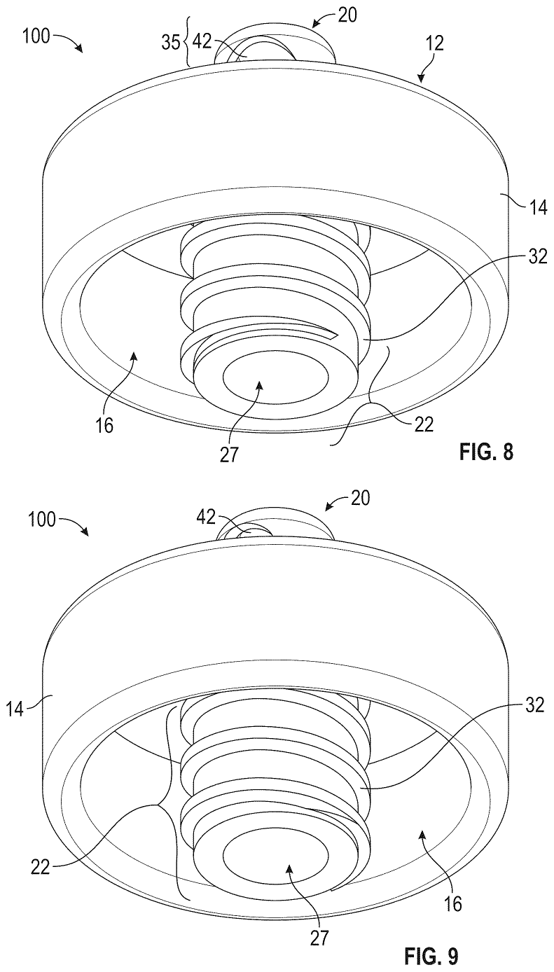

As shown in FIGS. 1-9, in accordance with embodiments of the present disclosure, an apportion structure 100 has a base portion 10 and an application portion 20. The base portion has an outer surface 12 and a sidewall 14 which together form a base cavity 16. In the embodiment shown, the base portion 10 is generally round and having a cylindrical shape. The sidewall 14 is generally perpendicular to the outer surface 12. The diameter of the outer surface 12 is greater than the outer height of the sidewall 14.

In the embodiments shown, the application portion 20 comprises a hollow tube-like structure extending through the base portion 10 generally perpendicular to, and preferably perpendicular to, the outer surface 12. The application portion 20 has a first open end 22 within the base cavity 16 and a second closed end 24 outside of the base cavity 16 which forms a nipple portion 35. The application portion 20 thereby forms an inner channel 27 which is open within the base cavity 16 and closed at the nipple portion 35. When in use, a gelatinous or viscous solution will flow through the inner channel 27 to the nipple portion 35 of the application portion 20.

The application portion 20 further contains attachment structures 32. In the embodiment shown the attachment structures 32 are along the outer surface 36 of the application portion 20 extending within the base cavity 16. In other words, the application portion 20 essentially create a male connection portion. In further embodiments, the attachment structures 32 may be on the inner surface of the application portion 20 extending within the base cavity 16, thus creating a female connection portion (described here but not seen in the drawings).

In the embodiment shown, the attachment structures are threads along the outer surface 36 of the application portion that extends within the base cavity 16. The attachment structures 32 may be any attachment structures that facilitate attachment of the apportion structure 100 to a contained volume of a gelatinous or viscous solution, whether directly or indirectly. For example, the attachment structures 32 may be configured to connect directly to a structure such as a syringe, vial, or other container that holds a contained volume of a gelatinous or viscous solution, or connect to such structures with an indirect connection, such as through pumps, plastic tubes, or rubber tubes, or any other structure that could hold or transport a gelatinous or viscous solution.

The inner channel 27 proceeds into the nipple portion 35.

The nipple portion 35 has a smooth outer surface 40 for insertion into a body cavity, such as a nostril or ear cavity, or injury site, such as a puncture or laceration. The nipple portion 35 contains at least one opening or port hole 42 through which the gelatinous or viscous solution can pass from the apportion structure 100 into a cavity or injury site. In the embodiment shown, there are three openings; however, in further embodiments, there may be more or fewer openings, including, for example, from 1, or 2, or 3 to 4, or 5, or 6, or 7, or 8, or 9, or 10 openings. In a particular embodiment, the nipple portion 35 contains from 2, or 3 to 4 or 5 openings.

Further, in the embodiment shown, each of the openings or port holes 42 is, preferably, identical in size and shape. As shown in the Figures, the openings or port holes 42 are generally oval-shaped, and preferably with the greatest dimension of the oval shape being generally perpendicular to the outer surface 12. In a particular embodiment, the openings or port holes 42 have a first dimension (short dimension) of from 3.3 mm, or 3.4 mm to 3.5 mm, or 3.6 mm, and a second dimension (long dimension) of from 4.3 mm, or 4.4 mm to 4.5 mm, or 4.6 mm. In the exemplary embodiment shown, the first dimension is 3.5 mm, or 3.5 mm+/−0.05 mm, and the second dimension is 4.5 mm+/0.05 mm. In still further embodiments, the openings or port holes 42 may have different geometries, such as, for example, round, square, bell-shaped.

With reference to the Figures, it will be understood that each opening or port hole 42 in the embodiment described is, preferably, identical, that is, has the same shape, size and orientation.

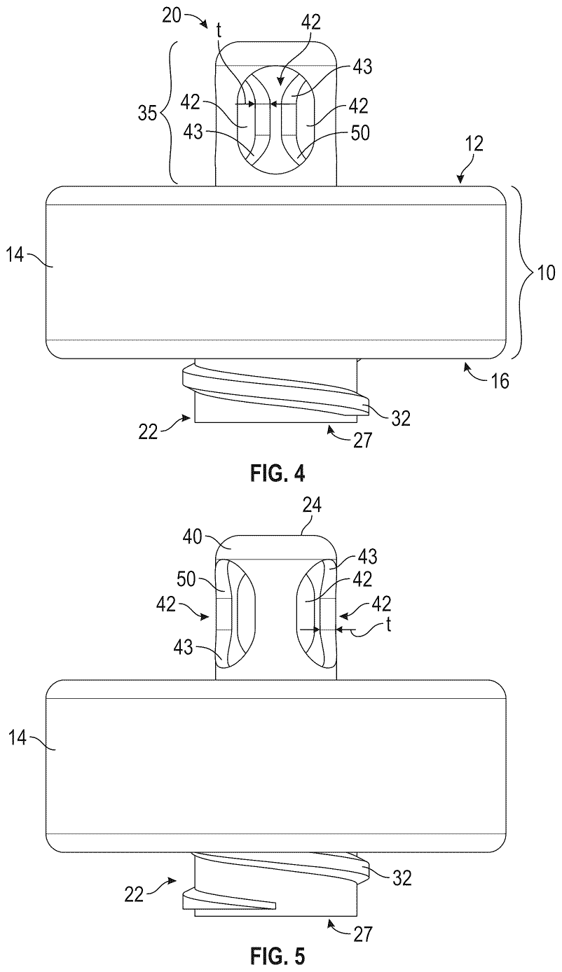

In the embodiment shown, the nipple portion 35 has a wall thickness t. The thickness forms the inner surface 50 of the openings or port holes 42. As shown in the Figures, and with particular reference to FIGS. 4 and 5, the inner surfaces 50 are smooth. A smooth inner surface is preferable to encourage consistent flow of gelatinous or viscous solution through the openings or port holes 42. The inner surfaces 50 of the openings or port holes 42 are also shown as having a width w that is approximately equal to, or equal to the wall thickness t at every point around the inner surface 50. In other embodiments, the all or portions of the inner surfaces 50 may be angled such that a portion of or all of the width of the inner surfaces are greater than or less than t. In some embodiments it may be beneficial to have inner surfaces 50 with a variable width around a given opening or port hole 42.

In an embodiment, the wall thickness t is from 0.8 mm, or 0.85 mm, or 0.9 mm to 0.95 mm, or 1 mm, or 1.1 mm. In an embodiment shown, the wall thickness tis 1 mm+/−0.1 mm. In the exemplary embodiment shown, the wall thickness tis 0.906 mm.

The outer surface 12 of the base portion 10 acts as a stop and ensures nipple portion 35 is inserted only to a fixed depth. The outside diameter of the nipple portion 35 is from 4 mm, or 4.5 mm to 5 mm, or 5.5 mm. The inside diameter of the inner channel 27 is from 3.0 mm, or 3.1 mm to 3.2 mm, or 3.3 mm. In a particular embodiment, the outside diameter of the nipple portion 35 is approximately, or is, 5 mm. In a particular embodiment, the inside diameter of the inner channel 27 is approximately, or is, 3.187 mm.

The nipple portion has a height from 5.8 mm, or 5.9 mm to 6 mm, or 6.1 mm, or 6.2 mm. In the exemplary embodiment shown, the height of the nipple portion is approximately, or is, 6 mm.

While not limited to any particular theory, it was unexpectedly discovered that an apportion structure as disclosed herein results in a generally even and gentle distribution of a gelatinous or viscous solution about the nipple portion. It is theorized that combination of one or more of (i) the number of openings, (ii) the shape of the openings, (iii) the smooth inner surface of the openings, (iv) the dimensions of the openings, and (v) the angle of the inner surface of the openings contributes to the even and gentle distribution of the gelatinous or viscous solution in the body cavity or injury site. In an embodiment, the apportion structure as at least one of:
(i) at least three openings;
(ii) at least one opening, wherein the opening is (or openings are) oval-shaped;
(iii) at least one opening, wherein the opening has (or openings have) at greatest dimension being perpendicular to the outer surface 12 of the apportion structure;
(iv) at least one opening, wherein the opening has (or openings have) a first dimension of from 3.3 mm to 3.6 mm and a second dimension of from 4.3 mm to 4.6 mm;
(v) at least one opening, wherein the opening has (or openings have) a smooth inner surface; and
(vi) at least one opening, wherein the opening has (or openings have) a inner surface having a width approximately equal to, or equal to, the wall thickness at every point around the inner surface 50.
   In an embodiment, the apportion structure has at least two, at least three, at least four, at least five, or all six of (i)-(vi).

In accordance with embodiments of the present disclosure, a method of applying a gelatinous or viscous solution to a body cavity or injury site is provided.

A first step comprises providing an apportion structure. In an embodiment, an apportion structure 100, such as in accordance with any of the embodiments or combination of embodiments described herein, is used to apply the gelatinous or viscous solution, such as a hair removal wax or an ointment, to a cavity, such as a nostril or ear cavity, or injury site, such as a puncture or laceration.

In a particular embodiment, the step of providing an apportion structure comprises providing an apportion structure wherein the apportion structure comprises a base portion having an outer surface and a sidewall which form a base cavity and an application portion forming a hollow channel having a central axis and extending through the base portion generally perpendicular to the outer surface, wherein the application portion has a first open end extending to the base cavity and having at least one attachment structure, wherein the application portion further has a closed nipple portion extending outward from the outer surface of the base portion, and wherein the nipple portion has at least three openings therethrough.

The method further includes a step of securing the apportion structure to a contained volume of the gelatinous or viscous solution using the at least one attachment structure. In such an embodiment, a contained volume of gelatinous or viscous solution is attached to the apportion structure 100 using the attachment structures 32.

In an embodiment, the volume of gelatinous or viscous solution is contained in a pump, rubber tube, plastic tube, syringe, or combination of such structures. In such embodiment, the step of securing the apportion structure to a contained volume of gelatinous or viscous solution using the at least one attachment structure comprises engaging, with the at least one attachment structure, a structure selected from the group consisting of a pump, a rubber tube, a plastic tube, a syringe, and combinations thereof.

The method further comprises the step of inserting the nipple portion into the body cavity or injury site. The nipple portion may be inserted as far as permitted by the apportion structure or only a portion of the amount permitted.

The method further comprises forcing the gelatinous or viscous solution through the hollow channel of the apportion structure, out the nipple portion, and into the body cavity or injury site. That is, once the nipple portion 35 is inserted into the desired cavity or injury site, the volume of gelatinous or viscous solution is forced though the apportion structure 100 and out the nipple portion 35 through the openings 42.

In an embodiment, the volume of gelatinous or viscous solution is contained in a syringe. Once the nipple portion 35 is inserted into the desired cavity or injury, the plunger is depressed, thereby causing the volume of gelatinous or viscous solution to be injected into the cavity or injury site via the openings 42. In such an embodiment, the step of forcing the gelatinous or viscous solution through the hollow channel and out the nipple portion comprises depressing a syringe plunger thereby causing the volume of the gelatinous or viscous solution to be injected into the body cavity or injury site via the at least three openings.

In an embodiment, the body cavity or injury site is a body cavity, and the method further includes the steps of allowing the gelatinous or viscous solution to set within the cavity while the nipple portion still engages the cavity and removing the apportion structure with set gelatinous or viscous solution from the cavity.

In an embodiment, the body cavity or injury site is a body cavity which is a nostril or ear cavity, and the method further includes the steps of allowing the gelatinous or viscous solution to set within the nostril or ear cavity while the nipple portion still engages the nostril or ear cavity, and removing (i) the apportion structure with set gelatinous or viscous solution from the nostril or ear cavity and (ii) cavity hairs captured in the set gelatinous or viscous solution.

In an embodiment, the gelatinous or viscous solution is a hair removal wax and the method further includes allowing the hair removal wax to set, firm or harden in the cavity which is a nostril or ear cavity. Once the gelatinous or viscous solution is set, firmed, or harden, it is removed by pulling to remove cavity hairs. In a particular embodiment, the apportion structure 100 remains in the cavity while the gelatinous or viscous solution sets, firms, or hardens. The apportion structure 100 thereby functions as a removal structure as well, because once the gelatinous or viscous solution is set, firmed, or hardened, the apportion structure 100 is pulled to remove the gelatinous or viscous solution from the cavity along with any cavity hairs.

In an embodiment, the gelatinous or viscous solution is a hair removal wax and the apportion structure is designed to be removed from the contained volume of gelatinous or viscous solution once the solution is applied through the openings or port holes 42. In such embodiments, only the apportion structure would remain in the cavity while the gelatinous or viscous solution sets, firms, or hardens. In further embodiments, the contained volume of gelatinous or viscous solution is designed to remain secured to the apportion structure, such as, for example, to provide a grasping portion for removal. In such embodiments, the apportion structure and contained volume of gelatinous or viscous solution are separated after removal of the set, firmed, or hardened gelatinous or viscous solution. In some embodiments, the method therefore includes the step of removing, after forcing the gelatinous or viscous solution through the hollow channel, out the nipple portion, and into the body cavity or injury site, the contained volume of the gelatinous or viscous solution from the apportion structure. In other embodiments, the step of removing the apportion structure form set gelatinous or viscous solution from the cavity comprises grasping, after allowing the gelatinous or viscous solution to set within the cavity while the nipple portion still engages the cavity, the contained volume of gelatinous or viscous solution.

In an embodiment in which the gelatinous or viscous solution is a hair removal wax, the apportion structure is designed to be disposable and discarded with any gelatinous or viscous solution and/or hairs removed from the cavity. In embodiments, the contained volume of gelatinous or viscous solution may be removed from the apportion structure 100 before disposal. In further embodiments, the apportion structure 100 and contained volume of gelatinous or viscous solution are provided together, such as, for example, in a “single use” configuration, and the entire assembly is designed to be disposed of after use.

From the foregoing, it will be appreciated that although specific embodiments or examples have been described herein for purposes of illustration, various modifications may be made without deviating from the spirit or scope of this disclosure. It is therefore intended that the foregoing detailed description be regarded as illustrative rather than limiting, and that it be understood that it is the following claims, including all equivalents, that are intended to particularly point out and distinctly claim the claimed subject matter.

It is specifically intended that the present invention not be limited to the embodiments, examples, and illustrations contained herein, but include modified forms of those embodiments or examples including portions of the embodiments or examples and combinations of elements of different embodiments or examples as com within the scope of the following claims.

What is claimed is:

1. An apportion structure comprising:
a base portion having an outer surface and a sidewall which form a base cavity; and
an application portion forming a hollow channel having a central axis and extending through the base portion generally perpendicular to the outer surface,
wherein the application portion has a first open end extending into the base cavity and having at least one attachment structure,
wherein the application portion further has a closed nipple portion extending outward from the outer surface of the base portion,
wherein the nipple portion has at least one opening therethrough; and
wherein the nipple portion has a wall thickness, and wherein an inner surface of each of the at least one opening has a width equal to the wall thickness of the nipple portion along at least a portion of the inner surface.

2. The apportion structure of claim 1, wherein the nipple portion has greater than one opening therethrough.

3. The apportion structure of claim 2, wherein the nipple portion has at least 3 openings.

4. The apportion structure of claim 1, wherein each of the at least one opening is oval-shaped.

5. The apportion structure of claim 4, wherein each of the at least one opening has a smooth inner surface.

6. The apportion structure of claim 1, wherein the nipple portion has a wall thickness, and wherein the inner surface of each of the at least one opening has a width equal to the wall thickness of the nipple portion along the entirety of the inner surface.

7. The apportion structure of claim 1, wherein the at least one attachment structure is configured to engage a structure holding a contained volume of a gelatinous or viscous solution.

8. The apportion structure of claim 7, wherein the at least one attachment structure is on an outer surface of the first end of the application portion.

9. The apportion structure of claim 7, wherein the at least one attachment structure is on an inner surface of the first end of the application portion.

10. The apportion structure of claim 1, wherein the at least one attachment structure is configured to engage a structure selected from the group consisting of a pump, a rubber tube, a plastic tube, a syringe, and combinations thereof.

11. The apportion structure of claim 1, wherein the base portion is generally round.

12. An apportion structure comprising:
a base portion having a circular outer surface and a sidewall extending at a length from the outer surface to form a base cavity, wherein a diameter of the circular outer surface is greater than a length of the sidewall; and
an application portion forming a hollow channel having a central axis and extending through the base portion generally perpendicular to the outer surface,
wherein the application portion has a first open end extending into the base cavity and having at least one attachment structure,
wherein the application portion further has a closed nipple portion extending outward from the outer surface of the base portion,
wherein the nipple portion has at least three openings therethrough, wherein each of the at least three openings has a smooth inner surface; and
wherein the nipple portion has a wall thickness, and wherein the smooth inner surface of each of the at least three openings has a width equal to the wall thickness of the nipple portion along at least a portion of the smooth inner surface.

13. The apportion structure of claim 12, wherein each of the at least three openings is oval-shaped.

14. The apportion structure of claim 13, wherein each of the at least three openings has a first dimension in a short direction of 3.3 mm to 3.6 mm and a second dimension in a long direction of 4.3 mm to 4.6 mm.

15. The apportion structure of claim 12, wherein the at least one attachment structure is configured to engage a structure holding a contained volume of the gelatinous or viscous solution.

16. A method of applying a gelatinous or viscous solution to a body cavity or injury site comprising:
providing an apportion structure, wherein the apportion structure comprises
a base portion having an outer surface and a sidewall which form a base cavity; and
an application portion forming a hollow channel having a central axis and extending through the base portion generally perpendicular to the outer surface,
wherein the application portion has a first open end extending into the base cavity and having at least one attachment structure,
wherein the application portion further has a closed nipple portion extending outward from the outer surface of the base portion, and
wherein the nipple portion has at least three openings therethrough,
securing the apportion structure to a contained volume of the gelatinous or viscous solution using the at least one attachment structure;
inserting the nipple portion into the body cavity or injury site;
forcing the gelatinous or viscous solution through the hollow channel and out the nipple portion and into the body cavity or injury site;
allowing the gelatinous or viscous solution to set within the body cavity or injury site; and
removing the apportion structure with set gelatinous or viscous solution from the body cavity or injury site.

17. The method of claim 16, wherein the step of securing the apportion structure to a contained volume of the gelatinous or viscous solution using the at least one attachment structure comprises engaging, with the at least one attachment structure, a structure selected from the group consisting of a pump, a rubber tube, a plastic tube, a syringe, and combinations thereof.

18. The method of claim 17, wherein the step of securing the apportion structure to a contained volume of the gelatinous or viscous solution using the at least one attachment structure comprises engaging, with the at least one attachment structure, a syringe containing a volume of the gelatinous or viscous solution, and wherein the step of forcing the gelatinous or viscous solution through the hollow channel and out the nipple portion comprises depressing a syringe plunger thereby causing the volume of the gelatinous or viscous solution to be injected into the body cavity or injury site via the at least three openings.

19. The method of claim 17, wherein the body cavity or injury site is a nostril or ear cavity, and further comprising the steps of:
allowing the gelatinous or viscous solution to set within the nostril or ear cavity while the nipple portion still engages the nostril or ear cavity; and
removing (i) the apportion structure with set gelatinous or viscous solution from the nostril or ear cavity and (ii) cavity hairs captured in the set gelatinous or viscous solution.

20. The apportion structure of claim 1, wherein the nipple portion has at least three openings, and each of the at least three openings is oval-shaped.

21. The apportion structure of claim 20, wherein each of the at least three openings has a wall thickness equal to the wall thickness of the nipple portion along the entirety of the inner surface.

22. The apportion structure of claim 1, wherein the at least one attachment structure is configured to engage a structure holding a contained volume of hair removal wax.

23. The apportion structure of claim 15, wherein the contained volume is a contained volume of hair removal wax.

24. An apportion structure comprising:
a base portion having an outer surface and a sidewall which form a base cavity; and
an application portion forming a hollow channel having a central axis and extending through the base portion generally perpendicular to the outer surface,
wherein the application portion has a first open end extending into the base cavity and having at least one attachment structure,
wherein the application portion further has a closed nipple portion extending outward from the outer surface of the base portion,
wherein the closed nipple portion has an outer diameter and an inner diameter, the inner diameter defining an inner channel,
wherein the outer diameter is from 4 mm to 5.5 mm and the inner diameter is from 3.0 mm to 3.3 mm, and
wherein the nipple portion has at least one opening therethrough.

25. The apportion structure of claim 24, wherein the nipple portion has a height from 5.8 mm to 6.2 mm.

26. The apportion structure of claim 25, wherein the nipple portion has a wall thickness defined as half of the difference between the outer diameter and inner diameter.

27. The apportion structure of claim 26, wherein the wall thickness is from 0.8 mm to 1.1 mm.

28. The apportion structure of claim 27, wherein the nipple portion has at least three openings therethrough.

29. The apportion structure of claim 28, wherein the at least three openings each have an inner surface having a thickness equal to the wall thickness along at least a portion of the inner surface.

30. The apportion structure of claim 28, wherein the wall thickness is from 0.9 mm to 1.1 mm, the outer diameter is from 4.5 mm to 5.5 mm, and the inner diameter is from 3.1 mm to 3.2 mm.
